(19) United States
(12) Patent Application Publication
(10) Pub. No.: US 2022/0175523 A1
(43) Pub. Date: Jun. 9, 2022

(54) STENT DEVICE FOR A PROSTHETIC HEART VALVE

(71) Applicant: T-Heart SAS, Paris (FR)

(72) Inventors: Alain Dibie, Paris (FR); Soad El Ghazouani, Paris (FR)

(21) Appl. No.: 17/606,713

(22) PCT Filed: May 15, 2020
(86) PCT No.: PCT/EP2020/063739

§ 371 Date: Oct. 26, 2021

(30) Foreign Application Priority Data
May 17, 2019 (EP) PCT/EP2019/062842

(51) Int. Cl. A61F 2/24

(52) U.S. Cl. CPC A61F 2/2418; A61F 2/2415; A61F 2230/0069; A61F 2002/9505; A61F 2230/0056; A61F 2230/0008; A61F 2230/005

(57) ABSTRACT

Replacing a defective atrioventricular heart valve, in particular a tricuspid valve, may include stent devices, prosthetic heart valves, delivery systems, and corresponding methods, which provide an improved fixation without distortion of the native anatomy. A stent device for a prosthetic heart valve has an axially extending mesh-shaped body, configured to fit an orifice and defining an inner channel as a passageway from a proximal to a distal end. At least three outer support arms extend from the distal end of the body towards the proximal end. Each support arm has a distal end first support region and a proximal end second support region. The second support region extends radially outwards in the deployed state. Each support arm has a flexible region between the first and second support regions, which is formed as an axially tapered section of the support arm and/or each support arm is tapered towards the proximal end.

### CROSS-REFERENCE TO RELATED APPLICATIONS

This application is the U.S. National Stage of PCT/EP2020/063739 filed May 15, 2020, which claims priority to PCT Patent Application No. PCT/EP2019/062842, filed May 17, 2019, the content of both are incorporated herein by reference in their entirety.

### FIELD OF THE INVENTION

The invention relates to the field of replacing a defective atrioventricular heart valve, in particular a tricuspid valve, including stent devices, prosthetic heart valves, and delivery systems, as well as methods for producing such stent device and methods for replacing a tricuspid valve or mitral valve using such stent device.

### BACKGROUND OF THE INVENTION

The blood circulation in mammals is primarily driven by the pumping function of the heart. Such cardiac function is provided not only to ensure that tissue is sufficiently perfused, but also to provide a de-carbonization and re-oxygenation of effluent blood after passing tissue. The human heart comprises two ventricles, the left ventricle and the right ventricle, which respectively pump blood through the vascular system via the aorta and through the pulmonary system via the pulmonary artery so as to provide a respiratory function and oxygenize the blood. The filling of said ventricles is enabled by the corresponding left atrium and right atrium, which are respectively connected to the pulmonary vein and the vena cava.

In order to provide a proper functioning of the atria and ventricles, the human heart has four heart valves. Two of these valves are called the atrioventricular valves, i.e. being located at the junction between the atrium and ventricle. The tricuspid valve is located between the right atrium and the right ventricle. The mitral valve, also known as the bicuspid valve, is located between the left atrium and the left ventricle. The other two valves are positioned between the ventricles and the vascular system and comprise a semilunar shape. The aortic valve separates the left ventricle from the aorta, and the pulmonary valve separates the right ventricle from the pulmonary artery.

The filling and ejection of the atria and ventricles follows in a highly synchronized regimen during the diastolic phase and systolic phase. However, the efficiency of the cardiac function is not only dependent on the complex innervation of the myocardial tissue, but also on the sealing efficiency of the atrioventricular valves. Such sealing efficiency may be impaired due to various pathological conditions, e.g. due to a functional pathology of the tricuspid valve, which pathology may be elusive, severe, and secondary to a significant dilation of the tricuspid annulus. More rarely, this pathology is due to a rheumatic or infectious valvular disease or to degeneration of a stenosing or leaking bioprosthesis.

A dysfunctional tricuspid valve may lead to tricuspid regurgitation, which is a common medical problem and is associated with significant challenges. For example, patients suffering from tricuspid regurgitation generally suffer from a chronically disfunctional fluid retention and have a low cardiac output. Furthermore, the annulus diameter may extend over 40 mm, such that anatomic landmarks between the right ventricle and right atrium are gradually lost, thereby impairing and complicating treatment, repair and replacement of the tricuspid valve.

More generally, replacement techniques of defective heart valves, in particular by a percutaneous route or by a minimally invasive route are established. However, such systems and techniques are primarily aimed at replacement of the mitral valve, which are not directly applicable to a replacement of the tricuspid valve. Among other distinctions, the right ventricle comprises a unique anatomy. The tricuspid annulus is only slightly fibrous by nature. Its dimensions are more ovoid while generally having a thinner structure compared with the mitral annulus. Furthermore, the dimensions of the tricuspid valve are substantially larger than the mitral valve. These differences may become even more prominent as a result of pathophysiological conditions causing structural alterations of the right atrio-ventricular anatomy in shape and dimensions with fluctuations in volume status and pulmonary pressure, such that the tricuspid annulus may dilate to a diameter beyond 40 mm, e.g. up to more than 50 mm, whereas the mitral annulus' dimensions are about 30 to 35 mm under pathological conditions. Said differences directly affect both the mechanical stability and the propensity for peri-prosthetic leaks, such that—in contrast to assertions in the literature—mitral valve replacement techniques are typically not applicable to the tricuspid valve.

The clinical practice pertaining to aortic valves is at present far advanced such that aortic valves are routinely replaced by percutaneous valves. Bioprosthesis models for the percutaneous mitral valve are also currently in the process of clinical evaluation. In contrast, clinical treatments involving a percutaneous orthotopic, i.e. in situ, bioprosthesis for tricuspid valve replacement are at a very early stage of development. Tricuspid valve replacement is also intricate, as that body site lacks generally body tissue volume holding a prosthetic device in place. Various techniques are based on prosthetic devices having a conical plug shape to facilitate the fixation. They generally exert outwardly directed radial forces that may further distort the anatomy at the right atrioventricular site. Alternative fixation-based techniques using the leaflets of the tricuspid valve are only adapted to a limited portion of the tricuspid valve while at the same time requiring a profile height of more than 30 mm, which is bulky, may increase the risk of dislodgement, and may impair blood flow.

For example, a prosthetic valve for a tricuspid valve is known from WO 2016/098104 A2, which comprises a flexible body with rigid ventricular stabilizers that engage the native leaflets of the tricuspid valve. Such arrangement exhibits limited support of and adaptability to the anatomical structures of the tricuspid valve and requires a clamping of the native leaflets, which may be detrimental for the remaining anatomical landmarks.

Furthermore, WO 2017/089179 A1 discloses an assembly for replacing the tricuspid atrioventricular valve, wherein supporting arms or fixing elements extend from a central portion of the mesh body. Such an arrangement requires a fixation at the central portion.

Accordingly, there is a need for devices, systems, and methods specifically adapted to the replacement of the tricuspid valve, which reduce the above problems and which provide an improved fixation without distortion of the native anatomy of the tricuspid valve. Such devices or systems may also improve support for mitral valve replacement.

### SUMMARY OF THE INVENTION

It is an object of the present invention to provide a stent device for a prosthetic heart valve which abrogates at least some of the above observations being undesirable for the clinical practice.

Accordingly, in a first aspect, a stent device for a prosthetic heart valve is suggested, which comprises a mesh-shaped body extending in an axial direction, wherein the body is configured to fit an orifice and defines an inner channel for providing a passageway from a proximal end to a distal end of the body. The stent device furthermore comprises at least three outer support arms, which extend from the body, i.e. from the distal end of the mesh-shaped body towards the proximal end, wherein each support arm comprises a first support region at the distal end, a second support region at the proximal end, and a flexible region therebetween. The flexible region of each support arm is formed as a tapered section of the support arm in an axial direction and the second support region of each support arm extends radially outwards in the deployed state.

The body of the stent device hence forms a supporting structure or core frame, which may be accommodated in an opening e.g. provided by the annulus of the tricuspid valve or mitral valve. The body is to be understood as not being resilient or allowing a deflection, yet may be deformable, such that the body and the stent device as a whole may transition between a collapsed and expanded or deployed state. For example, the stent device may be formed from a metallic memory material, e.g. braided nitinol, such that the shape of the body may be predefined, e.g. by heat forming, and/or that the stiffness of the body may vary dependent on and/or at intervals of temperature between, preferably, for example, 0° C. and body temperature, while the dimensions of the body may vary.

In contrast, the flexible region is to be understood as being resilient or allowing deflection, such that the second support region may e.g. be bent, deflected, folded, or pivoted with respect to the first support region via the flexible region.

Furthermore, the mesh-shape may e.g. be formed as a lattice or as a plurality of polygonal or ellipsoid cells that are connected either directly to each other or via struts. For example, the mesh-shape may be comprised of a plurality of polygons that are arranged laterally to each other to form a closed structure and which comprise a plurality, e.g. two, three, or more polygons in an axial direction, so as to essentially form a honeycomb structure. The plurality of polygons or cells in the axial direction may be connected via corresponding struts, preferably having equal strut length. Preferably, the mesh-shape of the body is formed by a lattice of a plurality of diamond-shaped cells that are directly connected to each other or are connected via struts, wherein the cells are preferably essentially equal in size and/or shape. The diamond shape has the advantage of providing a substantially equal stress and strain resistance in the axial and circumferential direction and that the amount of deformation and strain applied during manufacturing may be reduced so as to increase the stability of the body.

In the deployed state, the axial or longitudinal direction of the body is oriented essentially sagittal while the radial direction is oriented essentially in a transversal direction. Accordingly, the terms proximal and distal are to be understood in the anatomical sense, so as to refer to a blood flow direction within the human heart. In other words, the proximal end may refer to the stent device's terminus positioned in the deployed state within the atrium (“atrial portion”), while the distal end may refer to the stent device's terminus positioned in the deployed state within the ventricle (“ventricular portion”) of the tricuspid valve and mitral valve, respectively. Hence, the inner channel or passageway is also oriented in the same direction, i.e. from a proximal to a distal end of the body, such that blood may flow from the atrium towards the ventricle through said passageway along its axis first passing the stent device's “atrial portion”.

The provision of at least three support arms has the advantage that the stability of the fixation is improved as compared to e.g. two support arms, such that the stent device may be properly supported upon deployment. The support arms extend from the stent body at the distal end, i.e. from the body's “ventricular portion” upon deployment and extend towards the proximal end, i.e. the “atrial portion”. The extension from the body hence enables each of the support arms to originate from the body at its distal end and is hence only attached to the body at its distal end.

Each of the support arms is hence supported only at one side at a free end of the body in the “ventricular portion”. That concept does not only ensure an improved supportive function, e.g. by providing an adaptable spring force extending through the entire support arm, but also enables omission of any requirement for central fixation at a middle portion of the body. Thereby, the support arms may exhibit an improved adaptation to the anatomical structure of the valve and valve annulus by providing an improved force distribution. Furthermore, such arrangement together with the flexible region ensures an improved flexibility, allowing the second support region to better conform to the anatomy due to the deflection in the radially outward direction. The support arm arrangement and the provision of the flexible region hence provide an improved interrelation or interaction between the first support region and the second support region.

Alternatively, or in addition to the flexible region, each support arm may be tapered towards the proximal end. In other words, a tapered section of each support arm may be provided starting e.g. from the distal end of the stent body, from the flexible region, or at a proximal tip end, i.e. at the second support region of the respective support arm. The tapering may be provided as an essentially triangular or conical shape and may be continuous towards the proximal tip end or be truncated by a rounded portion at the proximal tip end. The continuous tapering, e.g. starting after the first support region, has the advantage that less strain may be applied during manufacturing, thereby increasing the stability while maintaining or providing a degree of flexibility of the support arm. Furthermore, this feature may prevent an entanglement during loading of the stent device in a delivery system, such that an entanglement, intertwining, interlocking, torsion, and/or twisting of the support arms is effectively avoided. At the same time, a spring function is maintained between the second support region and the first support region, such that a conformity of the support arms to the anatomy of the valve and valve annulus.

In addition, the extension of the support arms from the distal end only allows for an improved stability, since a separate fixation at e.g. a central or middle portion is not required anymore. Furthermore, insertion and positioning of the stent device at the desired anatomical position prior to deployment, i.e. prior to expanding the stent device is facilitated, as this allows the distal part to be positioned first and the stent device to be subsequently expanded towards the proximal end, while withdrawing the delivery system. By the same token, this arrangement allows for further adjustments during deployment, which may be limited whenever supporting arms are provided at a central or middle region.

Although the stent device may be adapted to be deployed at various regions of the atrioventricular heart valve, the mesh-shaped body is preferably configured to fit an annulus of the heart valve, wherein the flexible region may be adapted to conform to the annulus. The first support region may be adapted to conform to the ventricular portion of the annulus, and/or the second support region may be adapted to conform to the atrial portion of the annulus.

Accordingly, the stent device may fit the tricuspid valve or mitral valve along an annulus of the respective valve. Preferably, the second support region, i.e. at the proximal end, of each support arm is configured to be arranged at the atrial portion of the annulus such that the radially outward extending portion may be oriented to conform to the atrial portion of the annulus. This conformation is facilitated by the flexible region, which allows a deflection of the proximal end of the support arm, such that the contact surface of the second support region to the atrial portion is increased. By the same token, the first support region, i.e. at the distal end, of each support arm is preferably configured to be arranged at the ventricular portion of the annulus such that the stent device is positioned by its flexible region at the radially most inwardly positioned portion of the annulus and is supported at either end of the annulus by the corresponding support regions of the supporting arms. The flexible region is hence preferably configured to adapt to the shape of the annulus and hence not only provides the required flexibility for the second support region, but also comprises a flexibility so as to conform to the anatomy of the annulus.

Thereby, the stent device provides an optimal adaptation to the geometry or anatomy of the atrioventricular valve and the corresponding annulus. Furthermore, by establishing two support regions for each single support arm, the body of the stent device is securely held in place without exerting a clamping force and without grasping the anatomical structures. Anchor features allowing for piercing tissue to secure the stent device are not required. Accordingly, the anatomy of a pathophysiological annulus and the atrioventricular valve is not (further) distorted and is not further compromised, such that the remaining cardiac function is not further impaired. The adaptation of the support arms to the anatomical structures also enables that the mesh body of the stent device is not required to be flexible, but may be formed as an essentially rigid structure, providing further stability for e.g. a valve assembly. At the same time, the inventive concept allows the stent device's body to exhibit smaller dimensions, as the stent device is not merely secured by a radially outwardly directed force.

The adaptation to the geometry or anatomy of the valve or the annulus is particularly advantageous in view of the more complex anatomy of the tricuspid valve. However, any such configuration may also be applied as a mitral valve replacement stent device, as it also advantageously contributes to the functionality of a mitral valve stent device.

The stent device's body may furthermore exhibit an essentially tubular or cylindrical shape. Such a geometry has the advantage that a less complex and/or more robust valve assembly may be implemented in the stent device. Still, a more versatile stent device is ensured, since orientation or positioning of the body is facilitated and is not dependent on a corresponding shape of the anatomical structure. Furthermore, an improved support at regions of the annulus having a smaller curvature is enabled, which maximizes the volume of the inner channel for improved blood flow.

For an increased contact surface and for reducing the amount of material and maintaining flexibility or adaptability of the support arms, each support arm may be advantageously formed as a closed loop. Although each support arm may generally be formed as a single extending element having e.g. a varying thickness in the axial direction and having a tapered or constricted section, a closed loop allows for increased stability with an even larger contact area or surface provided by the first and second support regions. For example, the closed loop may have a larger width at the distal end and/or proximal end compared with the flexible region. By the same token, the closed loop may typically be provided at a position distal from the flexible region, such that only the flexible region and the second support region are formed by the closed loop. Alternatively, the closed loop may be provided at a position distal from the first support region yet between the distal end of the body and the first support region of the support arm.

Preferably, the closed loop extends beyond the proximal end of the body. It may comprise a rounded (most) or tapered proximal end. The extension beyond the proximal end of the body has the advantage that the dimensions of the body may be kept at a minimally required size so as to reduce potentially bulky stent components protruding to the respective regions of the atrium or ventricle. Hereby, a larger contact area may be provided at the atrial portion of the annulus, thus further improving support.

By the same token, a rounded or tapered proximal end avoids any sharp surfaces edges or tips potentially piercing tissue of e.g. the atrial region, while simultaneously providing a larger contact area, e.g. compared with a pointed or spiked proximal end. Furthermore, the rounded shape increases the stability of the closed loop and reduces the risk of separation or breakage of the loop elements. For example, the closed loops may have an essentially petal shape, typically at the proximal end, so as to ensure the required adaptability or conformity to the corresponding anatomical structure.

The closed loop may furthermore define a profile having a convex portion and a concave portion in a longitudinal section of the support arm, wherein the convex portion is defined by the first support region. Preferably, the concave portion is adjacent to the convex portion.

For example, the first support region, which is arranged at the distal end, may be formed as a rounded shape that may conform to a ventricular portion of the annulus, while the concave portion may be formed, at least in part, by the flexible region. The flexible region or tapered section may e.g. be provided at a proximal part of the convex portion, starting in particular at an infliction point of the convex portion, such that the concave portion at least in part coincides with the tapered section. Thereby, the concave portion may comprise a degree of flexibility, which further facilitates the conformation to e.g. the annular anatomy and the interaction with the deflecting second support region at the proximal end.

Furthermore, a radial extension of a radially outermost point of the convex portion may be larger than a radially innermost point of the concave portion and/or a radially outermost point of the convex portion may radially be positioned between a radially innermost point of the concave portion and a proximal tip of the second support region.

In other words, a proximal tip end of the second support region may have the largest radial extension of each support arm while the first support region is arranged so as to extend beyond a concave portion directly adjacent to the convex portion. Accordingly, rather than a conical or frusto-conical extension of the support arms, a shoulder may be formed by the convex portion and the adjacent concave portion, which provides an improved spring function of the support arm and an improved adaptation to the annulus and ventricular anatomy. Furthermore, such configuration provides that the supporting regions of each support arm have a wider extension, such that a large surface area interacting with the annulus region is achieved in the deployed state and forces acting upon the stent device may hence be better diffused via the support arms.

Alternatively, the flexible region may overlap at least in part with the convex portion, e.g. may start at a proximal end of the convex portion and may extend beyond the distal end of the concave portion. In other words, the flexible region may at least in part overlap with the first support region and may not extend beyond the radially outwardly extending portion of the second support region.

Preferably, the profile is formed as an inverted S-shape, sine wave shape, N-shape, or M-shape in an axial direction and/or in a radial direction. Such a shape allows for a seamless transition between the varying regions and allows an optimal adaptation to the anatomical shape of the tricuspid valve or mitral valve, in particular to the corresponding annulus. Furthermore, such shape may comprise a second convex portion, which may be formed by the second support region. The stent device is biased in and may thus be positioned within the annulus and may be supported at either end of the annulus by the corresponding convex portions formed by the support regions of the supporting arms preferably without exerting a clamping or gripping force.

In addition, the convex and concave portions are not required to exhibit a symmetric profile and may have different curvatures, e.g. one or both convex portions may have an asymmetric profile, wherein a curvature at the “ventricular portion” or distal end of the stent device may e.g. be smaller or steeper compared with the curvature of the concave portion and/or the curvature of the “atrial portion” or proximal end of the stent device. Such steeper curvature, e.g. resembling a curvature of an N-shape or M-shape, may e.g. facilitate that the stent device is secured at the ventricular portion of the annulus, even in case of limited tissue volume, while the larger curvature, e.g. resembling an S-shape, at the “atrial portion” or proximal end may be foreseen so as to cover a larger atrial area or portion to provide a better distribution of the support and/or improved sealing.

Although the above particular shapes have been described in view of configurations having support arms formed as closed loops, these shapes are not limited to closed loop configurations and may also be implemented in embodiments having support arms formed as a single extending elements having e.g. a varying thickness in the axial direction.

Each support arm may be linked to the mesh body via at least one, preferably two, linking arm(s) formed by a curvature of the first support region. For example, such curvature may be formed by a convex portion originating from the distal end of the stent device's body. However, it may also be formed as a curvature independent of the presence of such convex first support region. In other words, each linking arm is provided as an extension of the body of the stent device and is typically formed integrally with the first support region of each support arm so as to form a continuous, e.g. single-piece structure. The curvature has the advantage that sharp edges may be avoided, such that the structural stability of the stent device and the connection between each arm and the body of the stent device is improved. In addition, the curvature ensures that the distal end of both the body and the support arms extending into the ventricular portion may be dimensioned smaller, so as to reduce the extension of parts of the stent device into the ventricle.

Furthermore, the curvature may represent a resilient element, such that a spring force biasing the support arms towards the anatomical structure of the atrioventricular valve, a less rigid support, and a higher adaptability to the anatomical structure are provided. The curvature may be adapted to the required spring force.

Advantageously, each support arm is linked to the body via two linking arms. Although linkage by a single arm may reduce the amount of material and may be sufficient in terms of structural stability, the provision of two linking arms increases the contact surface and force distribution of the first support region, e.g. with the ventricular portion of the annulus. In addition, such a “two linking arm” concept may prevent a rotation or lateral deflection of the support arm by providing two anchoring or fixation points at the body of the stent device.

The curvature of the linking arm may exhibit an angle of more than 90° and/or may define a rounded shoulder, wherein said shoulder preferably has a distal or first radius, e.g. at a distal end of the stent body, and a second or proximal radius, e.g. at a proximal portion of the shoulder, which may be proximal of and/or spaced apart radially and/or laterally from the distal radius, wherein the distal radius of the shoulder is larger than the proximal radius of the shoulder. As outlined in the above, the wider shoulder portion, together with the angle of the curvature, which may e.g. be formed by a convex portion and optionally an adjacent concave portion, has the advantage that the support arm may better adapt to the annulus region and ventricular anatomy and forces acting upon the stent device and the support arms may be better diffused, e.g. by providing an improved spring function established by an improved interaction with the second support region. Furthermore, the larger radius at the distal end of the rounded shoulder reduces the likelihood of fractures occurring between the support arm and the stent body, thereby increasing the stability and robustness of the stent device, while the smaller radius at the proximal end of the rounded shoulder maintains a sufficient support for the annulus region.

Furthermore, the particular configuration of the curvature or shoulder portion provides that the likelihood of entanglement with native chordae present at the ventricular portion is increased, such that an improved fixation and hence stability of the stent device may be achieved.

Accordingly, instead of extending from the body in an axial or radially outward direction, the curvature may initially extend radially inwardly, so as to form an additional rounded portion or an initial concave portion. This has the advantage that an even more flexible support is provided, such that an even lower force may be exerted by the first support region on the corresponding anatomical structure. In addition, by avoiding sharp edges, the risk of breakage or disconnection of the support arm from the body of the stent device is further reduced and tissue damages are essentially prevented.

As described in the above, provision of at least three support arms allows for the stability of the fixation to be improved as compared to e.g. two support arms, such that the stent device may be properly supported upon deployment. To further increase the support of the stent device into the orifice or annulus and to provide a further adaptability to the anatomical structure, the stent device may comprise a multitude or multiple of two and/or three support. e.g. 4, 6 or 8 arms adapted to a tricuspid valve or mitral valve.

Alternatively, also an uneven number of support arms may be provided, preferably 5, 7, or 9 support arms. Thereby, an improved adaptability to the anatomy of the annulus region and/or the valve assembly to be inserted into the body of the stent device may be ensured.

Preferably, the stent device may comprise six support arms. The provision of six support arms may provide a configuration for e.g. a tricuspid valve, which natively comprises three cuspids or valve leaflets, such that each pair of support arms may correspond to a region of the tricuspid valve comprising a respective leaflet. Accordingly, six support arms may increase both the stability and the adaptability of the stent device for the tricuspid valve.

Six support arms may also be suitable for a mitral valve, also known as bicuspid valve, natively comprising two leaflets. Three support arms may be arranged to correspond to a region of the mitral valve comprising a respective leaflet.

Alternatively, the stent device may also comprise e.g. four support arms, wherein the support arms are asymmetrically arranged so as to conform to the shape of the tricuspid valve. By the same token, four support arms may also facilitate a configuration for mitral valve replacement, wherein each pair of support arms is associated with a corresponding mitral valve region comprising a respective leaflet. Instead of a single pair of support arms, the stent device may also comprise e.g. eight support arms for e.g. a mitral valve or nine support arms for e.g. a tricuspid valve to accordingly associate four and three support arms to each native leaflet, respectively.

The circumferential spacing between the support arms may be adapted to a tricuspid valve or mitral valve and/or may be asymmetric or symmetric.

As described above, three support arms may e.g. be arranged so as to be associated with a respective native leaflet of a tricuspid valve, for example by providing an essentially equidistant spacing, but may also be asymmetrically arranged, such that e.g. a single support arm is associated with a first native leaflet and a pair of support arms is associated with a second leaflet, for example, in the case of a mitral valve configuration. Said arrangement may hence depend on the anatomical structure, e.g. of the annulus, and may further depend on the valve assembly to be used and/or the orientation of the stent device.

To provide further structural stability of the stent device, the mesh shape of the body may exhibit a diamond shape, or a droplet shape or an essentially oval shape. Hence, the cells may also comprise a rounded shape instead of a polygonal or honeycomb shape, which may facilitate the expansion and collapsing of the body of the stent device. Furthermore, a more even or balanced distribution of a biasing force in a radially outward direction is enabled, when the stent device is deployed. The stent body may thus be less susceptible to structural changes due to tensile or compressive forces acting on the stent device in an axial direction. The droplet shape or essentially oval shape allows for a more seamless transitioning between the cells and/or struts and hence avoids sharp edges.

In order to reduce the extension of the body of the stent device to the atrium, a portion of the proximal end of the stent body may extend radially outwards. Preferably, said portion of the proximal end of the body extends between 70° and 110° with regard to the axial direction of the body.

The bulkiness of the proximal end of the body extending into the atrium is reduced. Also fluidic sealing and supporting stability of the stent device is increased, e.g. by foreseeing the proximal end of the body to be adjacent to or in contact with the proximal end of the second support region of the support arms. For example, the proximal end of the body may be deflected at an angle essentially perpendicular to the longitudinal axis of the stent device, such that said proximal end is essentially aligned with the atrial portion of the annulus. Furthermore, insertion of e.g. a valve assembly is thus facilitated by providing a chamfer, while a reduced profile is presented, such that the blood flow is not or at least not significantly disrupted.

Said portion of the proximal region of the stent body is furthermore preferably defined by a plurality of second closed loops, which are preferably arranged in a circumferentially staggered formation with regard to the support arms arranged at the distal end. Accordingly, overlap of e.g. the closed loops of the support arms and the second closed loops is avoided on the one hand, on the other hand, the area or size of the atrial portion of the annulus that is supported by the proximal end is increased. The second closed loops may extend radially outwardly together with a portion of the proximal end of the body, e.g. by ¼ to ½ or ⅓ of the length of the ultimate cell or strut line at the proximal end of the body.

For example, the second closed loops may typically be dimensioned, such that they are smaller than the closed loops of the support arms, so as to fit into a distal end of two adjacent second support regions of two support arms, e.g. fitting between the second support region and the flexible region. According to another example, the second closed loops may be dimensioned such that they are essentially equivalent or larger than the closed loops of the support arms. Alternatively, the second closed loops may be arranged in between two adjacent second support regions of two support arms. Such a staggered structure may furthermore ensure sufficient support for the stent device at its “atrial portion”, even provided that one or more of the radially outwardly extending portions of the support arms do not perfectly conform to the anatomical structure, e.g. of the annulus. However, the second closed loops may also be arranged in a partial circumferential staggered formation or in an unstaggered or essentially overlapping formation with regard to the support arms arranged at the distal end.

By defining the support arms as an extension of the distal end of the body, a high degree of structural and mechanical stability is ensured.

The portion of the proximal end of the body extending radially outwards may furthermore comprise at least one eyelet for securing the stent device to a delivery system. Preferably, said portion comprises at least two eyelets with two eyelets being optionally arranged at opposing ends in a radial direction of the stent body. Each of the eyelets is arranged at a respective second closed loop, preferably at every second or third second closed loop and/or at the proximal end or radially outermost end of the second closed loop. In other words, the one or more eyelets may be arranged at a proximal tip end of the stent body, which is defined by the second loops flaring radially outward. To facilitate fixation, each of the eyelets may comprise an essentially circular or rounded shape, such that fixation and deployment are facilitated and do not require a particular relative orientation between the eyelets and the delivery system.

Furthermore, the body and the plurality of support arms may be formed as a single piece and/or as a wire frame. Accordingly, the body and supporting arms of the stent device may not only be formed of the same material, but may be formed without any connecting parts, such that the stent device is less susceptible to breakage, dislodgement and/or production errors.

By such an embodiment, the stent body, supporting arms, different cells of the mesh, and different regions of the support arms may be linked by a continuous and seamless transition. The body and support arms may hence be formed as integral parts of the stent device, wherein the stent device is formed as a single piece may be manufactured by laser cutting techniques, thereby significantly improving the structural integrity of the stent device.

To further improve fixation to the native tissue, in particular to improve the hold of the assembly in translation and/or rotation, the stent device may comprise at least one fixing element as disclosed in WO 2017/089179. In one embodiment, a fixing element has the form of a “racket” or a “loop” or a “tab”. In one embodiment, a fixing element is made of nitinol. A fixing element has a height in the order of 8 to 10 mm and a length of from 10 to 12 mm. In one embodiment, one or more of these optional fixing elements are joined to one or more support arms, more particularly in the flexible region thereof and open only on the atrial side. In one embodiment of the invention, a single fixing element is used. In one embodiment of the invention, two fixing elements are positioned symmetrically.

To accommodate the stent device to the anatomical dimensions of pathologic annulus regions and avoid detrimental engagement with the remaining native tissue as far as possible, the body or the passageway defined by the body preferably comprises an inner diameter of between 29 mm and 36 mm, preferably of about 30 mm or of about 35 mm. Such dimensions furthermore have the advantage that deployment and any potential further medical applications in the annulus region are facilitated.

In addition to the body and the support arms, the stent device may comprise a cuff to prevent blood leakage and/or regurgitation. Accordingly, at least the proximal end of the supporting arms, typically the second support region, and/or the proximal end of the outer body may be covered by a foil of a liquid impermeable or semi-impermeable material so as to form a cuff between the support arms and the body and/or between the support arms. Alternatively, such foil may cover at least the second support region and/or the distal end of the outer body to form a cuff between the support arms and the body and/or between the support arms.

The foil of a liquid impermeable or semi-impermeable material may hence represent a covering material, which typically limits, in particular in case of the semi-permeable material, or at least essentially prevents blood passage or blood reflux through the respective elements of the stent device outside the central (tubular) passageway. It thus seals the zone or contact area between the anatomical structure of e.g. the annulus and the body or inner channel of the stent device. Such a foil or covering may enhance sealing between the stent device and the environment at the deployment site. It also may enhance migration resistance of the deployed device by appropriate foil features, e.g. by suitable surface roughness.

The foil, e.g. made of a flexible sheet-like material, may be made of a natural or a synthetic material. For example, the material can include natural tissues such as, bovine, porcine, ovine, or equine pericardium, wherein the tissues are preferably chemically treated using glutaraldehyde or formaldehyde or triglycidylamine (TGA) solutions or other tissue crosslinking agents. Alternatively, the foil may at least comprise a synthetic material, such as a fluoropolymer, e.g. polytetrafluoroethylene (PTFE) or an expanded polytetrafluoroethylene (ePTFE) polymer, a polyester such as PET (polyethylene terephthalate), a silicone, a urethane, other biocompatible polymers, DACRON®, copolymers, or combinations and sub-combinations thereof. According to a preferred embodiment, the impermeable or semi-impermeable materials is a low-porosity woven fabric, such as polyester, DACRON® fabric, or PTFE.

Furthermore, the foil material may be modified by one or more chemical or physical processes that enhance certain physical properties of the foil material. For example, a hydrophilic coating may be applied to the foil material to improve the wettability and echo translucency of the foil material. Alternatively, or in addition, the foil material may be modified with chemical moieties that promote or inhibit one or more of endothelial cell attachment, endothelial cell migration, endothelial cell proliferation, and resistance to thrombosis. Also, the foil material may be modified by covalently attached heparin or may be impregnated by one or more drug substances that are released in situ, e.g. by a controlled release mode with e.g. the controlled release formulation being coated onto one or both sides of the foil.

The cuff established by the foil has the advantage that peri-prosthetic leaks at the contact area of the stent device and the native tissue (e.g. the annulus) may be reduced or even prevented. Preferably, the foil is arranged so as to cover the height of the annulus and the proximal end of the body on the “atrial portion” or proximal end of the stent device, typically covering also the second support region. The foil may be fixed to the stent device by means of e.g. sewing, gluing, or heat forming.

The body of the stent device furthermore preferably comprises at least two or at least three fixation means or windows or sites for receiving and fixing a valve assembly within the stent body's tubular passageway. Such windows or sites may e.g. be formed by corresponding struts between the cells or supporting the cells of the mesh-shaped body, such that the stent device does not require additional components. Said windows or sites are integrally formed with the body of the stent device. Alternatively, such windows may also be formed by corresponding portions of cells. The windows are preferably arranged circumferentially with spacings separating each of them. They correspond to the valve assembly, i.e. correspond to the envisaged leaflet arrangement of the particular valve, e.g. a tricuspid valve or mitral valve.

Said fixation means or windows may hence indicate the intended region for the valve assembly, such that a surgeon is assisted when placing of the valve assembly. The fixation means or windows may provide a matching geometry with the valve assembly, so as to provide a positive locking, e.g. in the case of a synthetic valve assembly, or may allow a fixation of a bioprosthesis, e.g. cusps or leaflets, e.g. by means of sewing or stitching.

Alternatively, the cells of the mesh-shaped body may be configured for receiving and fixation of a valve assembly, such that a valve assembly may be directly attached or fixed to one or more cells of the mesh-shaped body. This has the advantage that a larger flexibility is provided for the attachment and securing of the valve assembly and that the body is adapted to a larger variety of valve assemblies without having to adapt the stent body configuration. Furthermore, such configuration may reduce the complexity and increase the stability of the stent body and stent device. Accordingly, the fixation of the valve assembly and the manufacturing and deployment of the stent device may be facilitated.

According to another aspect, a prosthetic heart valve is disclosed comprising a stent device as described above and further comprising a valve assembly arranged within the inner channel and/or at a proximal or distal end of the body and being secured to the stent body by means of fixation means or windows. For example, the prosthetic heart valve may be configured for replacing a tricuspid valve or a mitral valve, e.g. by means of the corresponding valve assembly, the configuration of the stent body, and/or the configuration of the support arms, as described in the above.

For example, the prosthetic heart valve may be configured as a tricuspid valve prosthesis, wherein the valve assembly is comprised of three cusps or leaflets. For example, the tricuspid valve prosthesis may comprise a bioprosthesis, wherein the three cusps are made from animal tissue, e.g. bovine or porcine pericardium that has been preferably previously treated with e.g. glutaraldehyde or formaldehyde or triglycidylamine (TGA) solutions or other tissue crosslinking agents. The three cusps or leaflets forming the valve assembly are configured for coaptation with each other and are linked to the body within the inner channel, or at a proximal or distal end of the stent body, by means of e.g. common sewing techniques. The bioprosthesis functions in the physiological direction of the blood flow arriving in the right atrium and injected in systole into the filling chamber of the right ventricle.

Also, the prosthetic heart valve may be configured as a mitral valve prosthesis, wherein the valve assembly is comprised of two cusps or leaflets.

Furthermore, the cusps or leaflets of the valve assembly may also be made of a synthetic fabric, wherein the fixation may also be provided by e.g. welding, gluing, a fixed link or spring, and/or flexible or partially rotary contact points. Example of synthetic leaflets are provided in U.S. Pat. No. 9,301,837.

According to another aspect, a delivery system is suggested, comprising a stent device as described in the above in a collapsed state.

For example, the delivery system may be configured as a catheter or sheath, which enables a percutaneous delivery and deployment to the atrioventricular region. The delivery system may define a lumen to receive the stent device and may comprise a control string, wherein the control string is slidably engaged with the stent device such that a tensioning of the control string can cause the stent device to contract, and a loosening of the control string can allow the stent device to expand. The stent device is hence reconfigurable between a low-profile delivery configuration or collapsed state for containment within the lumen and an expanded or deployed configuration.

The invention further relates to a kit of part comprising a delivery system (e.g. a catheter or sheath) which enables percutaneous delivery and deployment to the atrioventricular region, and a stent device as described above.

The invention further relates to catheter or sheath comprising a collapsed stent device as described above.

According to another aspect, a method for replacing a tricuspid valve or mitral valve is disclosed, comprising the steps of:

providing a stent device as described above in a collapsed state in a delivery system, preferably a catheter or sheath,

percutaneously introducing the stent device into a tricuspid valve or mitral valve region of a patient via said delivery system, such that the distal end of the stent body is at a ventricular portion and the proximal end of the stent body is at an atrial portion and the body and support arms straddle the annulus, and

deploying the stent device by expanding the stent device, such that the flexible region conforms to the annulus and the proximal ends or second support regions of the outer support arms conform to the atrial side.

In order to provide a valve function, the method may further include fixing a valve assembly to the inner channel and/or at a proximal or distal end of the stent body, wherein said fixing may either be preferably performed prior to collapsing of the stent device or may be performed using a delivery system after deployment and expansion of the stent device in the atrioventricular valve zone.

According to another aspect, a method of producing a stent device as described above is disclosed, comprising the steps of:

laser cutting the body and support arms from a metallic memory material;

heat forming the body and support arms, so as to provide a predefined shape of the body and support arms; and optionally

collapsing the body and support arms.

The metallic memory material may e.g. be braided nitinol. Furthermore, the collapsing of the body and support arms, or of the stent device as a whole, is optional and may e.g. be required for transportation purposes or for assembly or packaging into a delivery system, e.g. a catheter or sheath.

Preferably, the stent device is made from a single piece. For example, the laser cutting may be performed on a single piece of braided nitinol, such that the support arms are formed as an extension of the distal end of the body, thus providing a high degree of structural and mechanical stability. In other words, the body and support arms of the stent device may not only be formed of a single material, but may be formed without any connecting parts as a single piece, such that the stent device is less susceptible to breakage, dislodgement, and/or production errors, thus e.g. a significantly improving the structural integrity of the stent device.

The inventive design allows to overcome any limits of the adaptability at the opposing ends of the fixing elements in the longitudinal direction of the assembly. Thus, the present design does not limit any adjustment of the positioning upon deployment of the assembly (as observed by the prior art), which may be due to opposing ends of the fixing elements, which may engage the anatomical structure, thereby potentially preventing further adjustments. Thus, the inventive design exhibits superior properties, which have not yet been addressed by prior art assemblies.

### BRIEF DESCRIPTION OF THE DRAWINGS

The present disclosure will be more readily appreciated by reference to the following detailed description when being considered in connection with the accompanying drawings in which:

FIG. 1 is a graphical representation of a prior art embodiment of a prosthetic heart valve at a ventricular portion of the tricuspid valve;

FIG. 2 is a graphical representation of a stent device according to the invention deployed around the anatomical structure of the tricuspid valve;

FIGS. 3A-3D are schematic representations of stent devices according to the invention after laser cutting and prior to heat forming;

FIGS. 4A and 4B are perspective views of a stent device according to the invention in an expanded state;

FIG. 5 is a schematic perspective view of a stent device according to the invention having an alternative proximal end of the body;

FIG. 6 is a schematic side view of the stent device according to FIG. 5;

FIG. 7 is a schematic representation of a stent device according to the invention having an alternative proximal end of the body comprising second closed loops;

FIG. 8 is a schematic representation of a staggering formation of the proximal end of the stent device according to FIG. 7;

FIGS. 9A to 9C depict different views of a staggering formation according to FIG. 7 with alternative second closed loops;

FIG. 10 is a schematic representation of a stent device according to the invention after laser cutting and prior to heat forming having eyelets; and

FIGS. 11A and 11B depict different views of a stent device according to the invention with an alternative support arm configuration.

### DETAILED DESCRIPTION OF PREFERRED EMBODIMENTS

In the following, the invention will be explained in more detail with reference to the accompanying figures. In the Figures, corresponding elements are denoted by identical reference numerals and repeated description thereof may be omitted in order to avoid redundancies.

In FIG. 1 a graphical representation of a prior art embodiment of a prosthetic heart valve positioned within a tricuspid valve is shown. Accordingly, a prosthetic heart valve is positioned within the tricuspid valve by means of a delivery system 42, e.g. a catheter or sheath. The prosthetic heart valve is positioned such that the body 12 or framework of the prosthetic heart valve is oriented in a longitudinal direction from a proximal end 16 to a distal end 17 of the tricuspid valve. Accordingly, upon deployment or expansion of the prosthetic heart valve, the support arms 18 of the prosthetic heart valve grasp the leaflets 27 of the tricuspid valve, thereby forming ventricular stabilizers. The support arms 18 are hence arranged at the ventricular side 28 of the tricuspid valve and are configured to provide a stabilization of the leaflets 27. After deployment of the prosthetic heart valve, the body 12 or framework of the prosthetic heart valve extends towards the proximal end 16, wherein the body 12 is formed as a flexible mesh body to conform to the anatomical structure of the leaflet 27 and to exert a radially outward force towards the leaflet 27, such that the prosthetic heart valve is held by the leaflets 27 while neither the support arms 18 nor the body 12 are actually in contact with the ventricular portion 28 or the atrial portion 30 of the annulus.

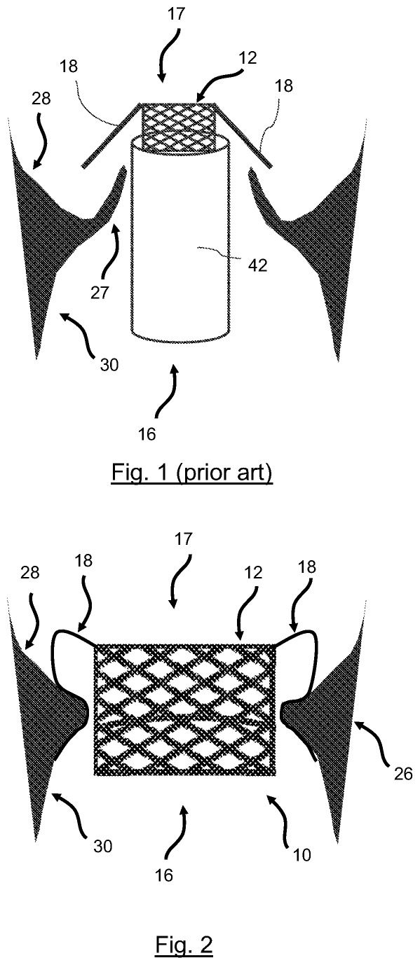

An embodiment of the stent device 10 according to the invention is depicted in FIG. 2 in a graphical representation, wherein the stent device 10 is deployed around the anatomical structure of the tricuspid valve corresponding to the anatomical structure according to FIG. 1. The stent device 10 is depicted in an expanded or deployed state, wherein a longitudinal axis of the stent device 10 and the body 12 is oriented along an axis defined by the proximal end 16 and the distal end 17 of the tricuspid valve. The body 12 is comprised of a mesh-shaped wiring made of nitinol and is formed as an essentially tubular body 12 having a cylindrical or circular shape.

A plurality of support arms 18 extend from the distal end 17 of the body 12. Although the sectional view depicts only two support arms 18, three or more, preferably six, nine, or twelve support arms 18 may be realized along a circumference of the distal end 17 of the body 12, e.g. at equidistant spacings or formed so as to be adjacently arranged to each other. The support arms 18 extend towards the proximal end 16 of the body 12 along an outer surface of the body 12. The inner surface is defined by an inner channel (not shown), which establishes a passageway for a blood flow from the proximal end 16 to the distal end 17, i.e. from the right atrium to the right ventricle of the heart during a systolic phase of the heart.

The support arms 18 furthermore comprise an S-shape or inverted S-shape in the longitudinal section of the stent device 10 and hence extend radially outwardly and inwardly along the longitudinal axis. Thereby, the support arms 18 define a first support region at the distal end 17 and a second support region at the proximal end 16, wherein the second support region extends radially outwardly at the proximal end 16 due to a deflection provided by a flexible region arranged between the first and second support region.

Accordingly, the shape of the support arms 18 and, in particular, the support regions allow the support arms 18 to conform to the ventricular portion 28 and the atrial portion 30 of the annulus 26. Furthermore, the flexible region is adapted to conform to the annulus 26 of the tricuspid valve. Thereby, the support arms 18 ensure that the stent device 10 is supported at opposing sides of the tricuspid valve and is fitted to the annulus 26 of the tricuspid valve, such that the stent device 10 is biased into the tricuspid valve region and in particular into the annulus 26 thereof.

Hence, the configuration of the support arms 18 allows the stent device 10 to be preferably secured to the annulus 26 without requiring invasive techniques such as sewing or stitching and without any clamping or grasping force or without exerting a radially outward force that potentially disrupts remaining anatomical landmarks and tissue. Instead, such configuration enables that the function of the body 12 of the stent device 10 is decoupled from the function of the support arms, such that the body 12 may be rigid, thus providing a stabile supporting structure or framework for e.g. a valve assembly.

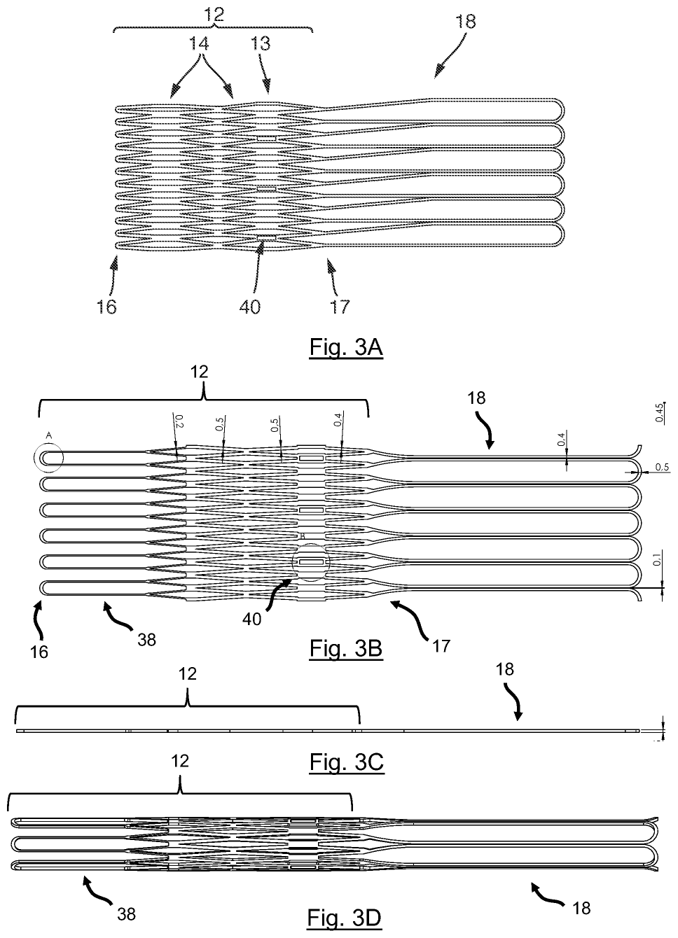

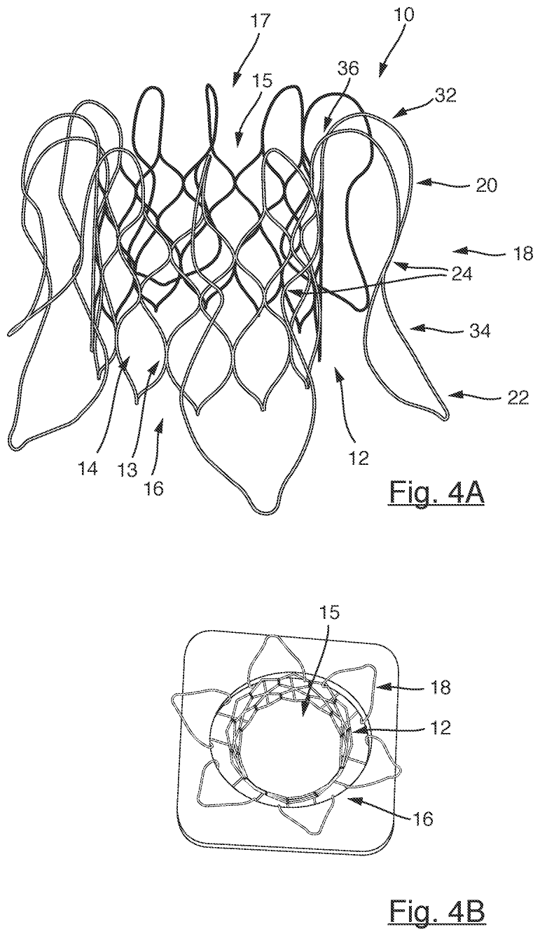

FIG. 3A is a schematic representation of a stent device, e.g. as depicted in the embodiment according to FIG. 2, wherein the stent device is depicted after laser cutting and prior to a heat forming process. On the left hand side, i.e. at the proximal end 16 of the stent device, the body 12 is depicted formed as a mesh shape. Said mesh is depicted to comprise two connected cells 14 arranged adjacently along the longitudinal axis of the stent device. The body 12 hence comprises a plurality of cells 14, which may be formed, e.g. by means of heat forming to a tubular or oval shaped structure so as to form a cylindrical shape defining an inner channel configured as a passageway for a blood flow.

Furthermore, the body 12 comprises three fixation means or windows 40, which are arranged at the distal end 17 of the body 12, wherein said windows 40 are formed by three corresponding struts 13 extending in the longitudinal direction. Accordingly, when the body 12 is formed into its predefined shape, the windows 40 are arranged in a circumferential manner at essentially equal spacing to each other. The windows 40 may e.g. be used to attach a valve assembly, e.g. synthetic or processed native cusps or leaflets to provide a required valve function adapted to the patient.

Although the windows 40 are depicted at the distal end 17 of the body 12, said windows 40 may also be provided at corresponding struts 13 at the proximal end 16 and the spacings between said windows 40 may vary. By the same token, the stent device preferably comprises at least three windows 40, e.g. for fixation of at least three cusps, e.g. for a tricuspid valve, but may also comprise more than three windows 40, so as to provide a physician or surgeon with more fixation possibilities.

According to the embodiment of FIG. 3A, the stent device furthermore comprises six support arms 18 extending from the distal end 17 of the body 12. After heat forming into a predefined shape, said support arms 18 extend towards the proximal end 16 at an outer surface of the body 12 and define two support regions and a flexible region, as described in view of FIG. 2 in the above. The support arms 18 are furthermore provided as closed loops, which are each linked to the body 12 via two linking arms, such that the structural and mechanical stability of the support arms 18 is increased. Furthermore, the support arms 18 comprise an essentially petal shape and which comprise a rounded proximal end after heat forming. Thereby, the support arms 18 provide an improved supporting function due to the increased contact area while the rounded proximal ends ensure that sharp edges and potential tissue damage are avoided and a risk of breakage of the proximal ends is reduced.

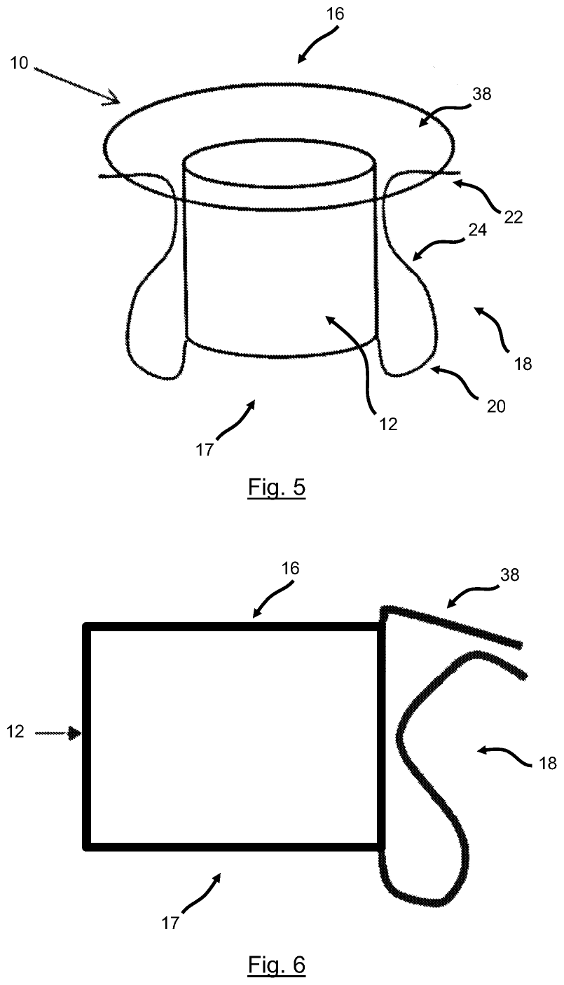

FIGS. 3B to 3D depict an alternative embodiment, wherein the proximal end 16 comprises second closed loops 38, which are arranged in a staggered formation with regard to the support arms 18. The proximal end 16 of the second closed loops may comprise a reinforcement formed by a thicker region to increase the linking strength between the legs of the second closed loops 38, as indicated on the left hand side of FIGS. 3B and 3D. The second closed loops 38 may be heat formed into e.g. a flaring shape, such that the second closed loops 38 extend radially outwardly, as explained in further detail in view of FIGS. 5 to 9 below.

Prior to heat forming, the stent device comprises an essentially flat shape extending in a longitudinal direction, as depicted by FIG. 3C. The stent device may be coiled along the longitudinal axis to form an essentially cylindrical body 12 with second closed loops 38 extending from the proximal end and support arms 18 extending from the distal end, as shown in FIG. 3D. Thereby, the body 12 of the stent device is given further structural stability and may be easily brought in its ultimate shape by heat forming the support arms 18 and second closed loops 38.

The embodiment according to FIG. 4A essentially corresponds to the embodiments according to FIGS. 2 and 3 and depict the stent device 10 after heat forming and in an expanded state. Accordingly, the stent device 10 also comprises a total of six support arms 18, formed as closed loops which extend from the distal end 17 of the body 12 via with two linking arms 36. As described in view of FIG. 2, the support arms 18 comprise a profile in the longitudinal section comprising an essentially inverted S-shape or sigmoidal shape, which are defined by a first support region 20 at the distal end 17, a second support region 22 at the proximal end 16 and a flexible region 24 therebetween. Said flexible region 24 is formed as a tapered section, e.g. formed by a constriction along the longitudinal axis of the stent device 10 so as to allow a deflection of the second support region 22.

The first support region 20 of each support arm 18 is formed as a convex portion 32, which at least in part defines a curvature forming each of the linking arms 36. The curvature ensures that the extension into the ventricular portion of the valve may be reduced, such that the blood flow is not disrupted and the stent device 10 is not brought in contact with any myocardial areas, which should not be contacted by the implanted stent device 10. The flexible region 24 is arranged around an infliction point of the convex portion 32 and is configured as a concave portion 34, extending into the second support region 22, which extends radially outwardly.

The convex portion 32, the concave portion 34, and the radially outwardly extending second support region 22 are thereby adapted to support a ventricular portion, an annulus portion, and an atrial portion of the tricuspid valve, respectively, such that each region provides an essentially matching geometry. The stent device 10 may hence be biased into the annulus of the tricuspid valve without invasively engaging or compressing the respective anatomical structures. Thus, disruption of the remaining anatomical structures may be effectively avoided.

Furthermore, the inner channel 15 is depicted in the embodiment according to FIG. 4A, which is defined by the mesh-shaped body 12 of the stent device 10. Accordingly, both the body 12 and the inner channel 15 comprise an essentially tubular and cylindrical shape, wherein the rigidity and size and dimensioning of the body 12 ensures that a maximum inner channel volume is provided so as to maximize the blood flow from the proximal end 16 to the distal end 17. The mesh body 12 is furthermore comprised of a plurality of cells 14 and corresponding struts 13, wherein the cells 14 comprise a droplet shape without sharp edges between adjacent cells 14, so as to further improve the structural integrity of the body 12.

When the stent device is in its deployed state, a gap may be formed between the outer surface of the body 12 and the support arms 18, as shown in the top view of FIG. 4B at the proximal end 16. Accordingly, the body 12 preferably exerts no radially outward force on the anatomical structures of the annulus and is only held on one side of the body 12 by the support arms 18, which may adapt both to the ventricular portion and the atrial portion so as to bias the stent device within the annulus. Due to the gap maximal flexibility and adaptability is ensured for the support arms 18 while at the same time ensuring that the anatomical shape is not deteriorated or compromised by the body 12 of the stent device.

An alternative configuration of the proximal end 16 of the body 12 is depicted in a schematic perspective view according to the embodiment of FIG. 5, wherein the stent device 10 comprises a body 12 having an alternative proximal end portion 38 of the body 12 extending in a radially outward direction, i.e. forming a flaring surface. Accordingly, the supporting arms 18, which extend from the distal end 17 of the body 12 towards the proximal end 16 of the body 12 may be brought into proximity or into contact with the proximal end portion 38 of the body 12.

For example, the support arms 18, which comprise, in addition to a first support region 20 at the distal end 17 and an adjacent flexible region 24, a second support region 22 at the proximal end 16, may deflect radially outwardly at the second support region 22 so as to be in contact with the radially outwardly extending proximal end portion 38. Thereby, the proximal end portion 38 may not only improve the sealing of the stent device 10, but may simultaneously facilitate the blood flow and/or an insertion into the inner channel of the body 12, e.g. by choosing an angle with regard to the longitudinal axis of the stent device 12 to define a chamfer. In addition, such arrangement may further increase the support of the stent device 10, by an additional surface that may be aligned with the corresponding atrial portion of the annulus or valve and to provide an additional supporting feature, should the second support region 22 not have the desired effect.

The embodiment according to FIG. 5 is furthermore schematically depicted in a side view according to FIG. 6. Here, the radially outwardly extending proximal end region of the body 12, is slightly tilted towards the outer surface of the body 12 and the support arms 18, e.g. at an angle between 70° and 90°. Such angle may e.g. be chosen to conform to the atrial portion of the annulus and may furthermore ensure an optimal sealing towards the proximal end 16 of the support arms 18. It is to be understood that other angles are possible and that the shape of the proximal end portion 38 is not restricted to the shape depicted in FIGS. 5 and 7, but may also comprise shapes corresponding e.g. to the mesh shape of the body 12.

Accordingly, in an exemplary embodiment, the stent device may comprise a proximal end portion 38 of the body comprising a plurality of second closed loops, as schematically depicted in FIG. 7. Said second closed loops are hence formed as extensions from the proximal end of the body, i.e. extensions from the mesh shape of the body, such that said second closed loops are connected to two adjacent cells 14 at the proximal end of the body. As indicated by the dashed line, the proximal end portion 38 that is extended radially outward, i.e. flares in a direction essentially perpendicular to the longitudinal axis of the stent device, comprises a portion of the last row of struts 13 of each of the last cells 14 at the proximal end of the body.

For example, the radially outward extending portion may comprise between ¼ and ½ of the last row of struts 13. In the embodiment according to FIG. 7, said portion comprises about ⅓ of the length of the last or ultimate row of struts 13 in the longitudinal direction of the stent device. Accordingly, depending on the anatomical structure corresponding to the pathophysiological condition of the patient, the second closed loops and the proximal end portion 38 may be at equal size with or sized smaller than the second support regions of each of the support arms.

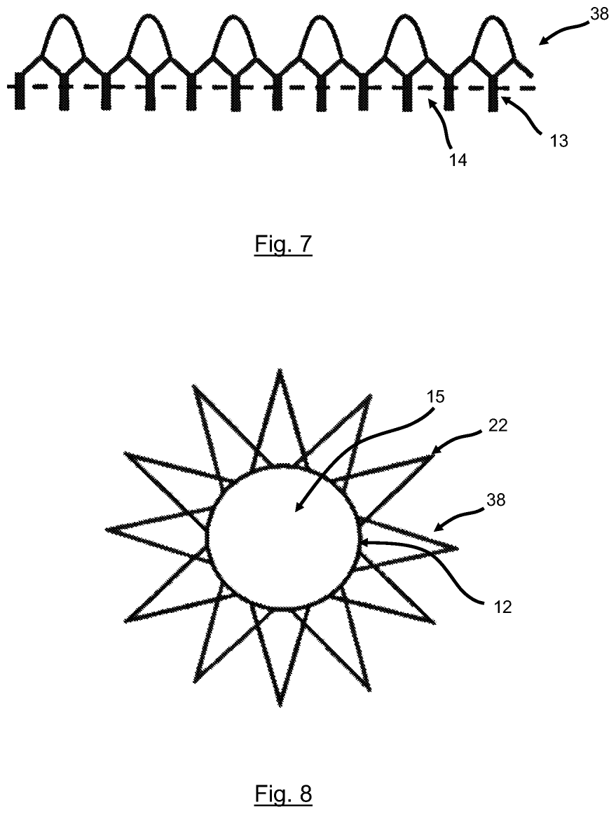

The proximal end portion 38 may furthermore be arranged at a staggering formation in view of the proximal end of the second support regions 22 of the plurality of support arms, as schematically depicted in the embodiment according to FIG. 8. Accordingly, the proximal end portion 38 may comprise a plurality of second closed loops extending from the proximal end of the body, e.g. as described in view of FIG. 7, which are deflected radially outwardly and are arranged between the second support regions 22 of each pair of adjacent support arms. Accordingly, the stent device may comprise a total of six support arms with corresponding second support regions 22 and a total of six second closed loops defined by the proximal end portion 38, which alternate each other in a circumferential manner along the circumference of the body 12 of the stent device. Also shown is the inner channel 15, which is hence not obstructed by the second support regions 22 and the proximal end portion 38. While the tips of the support regions 22 may be in the form of a triangle forming an acute angle at the end region, a rounded tip (not depicted by FIG. 8) may be more preferred.

Alternatively, the proximal end portion 38 may be provided in an alternative staggering configuration, e.g. when the number of second closed loops and the number of support arms do not match. For example, the proximal end portion 38 may comprise only three second closed loops, such that the second closed loops are arranged only between every second pair of adjacent second support regions 22. By the same token, the proximal end portion 38 may comprise a larger number of second closed loops, which are dimensioned smaller than the second support regions 22 of the support arms, such that e.g. two second closed loops are arranged between each pair of adjacent second support regions 22. It will be obvious to a person skilled in the art that the above number of second closed loops and second support regions 22 are for illustrative purposes only and are not limiting to the embodiments. In other words, other arrangements having a higher number of support arms or having a number between three and six support arms are possible and within the scope of the embodiments.

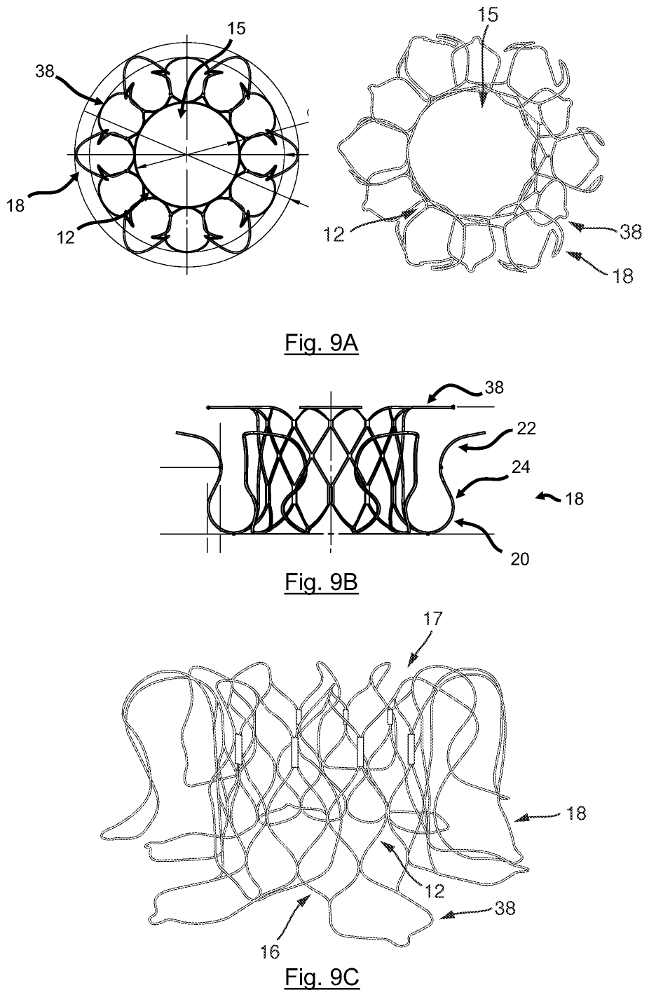

In the embodiment depicted in FIGS. 9A to 9C an alternative staggering formation of second closed loops 38 and support arms 18 is shown, wherein the second closed loops 38 are dimensioned such that they are similar to the closed loops of the support arms 18. Accordingly, as e.g. shown in FIG. 9A in the schematic top view (left) and perspective top view (right), the second closed loops 38 extend radially outwardly and may comprise a sectional surface area that is similar to the sectional surface area of the support arms 18. That holds, even though the second support region 22 of the support arms 18 may extend further radially outwardly, as also shown in FIGS. 9B and 9C, which represent a side view and a perspective view of the embodiment, respectively.

Furthermore, FIGS. 9B and 9C show a gap, which may occur between the support arms 18 and the body 12 of the stent device, in analogy to the embodiment according to FIG. 4B. Thereby, a tolerance is ensured between the support arms 18 and the body 12. Resilience of the support arms 18 is thus increased. In other words, as shown e.g. in FIG. 9B, the gap between the S-shape of the support arms 18, i.e. the convex and concave region in the longitudinal section, and the body 12 of the stent device may vary at least partially or sectionwise, so as to accommodate the stent device in the annulus, thereby adapting its to the anatomical structure thereof.

In addition, the flaring second closed loops 38 ensure that a direct contact between the body 12 and the anatomical structure may be avoided, such that the body 12 does not exert a radially outwardly directed force on the annulus. Potentially adverse forces that are detrimental for the remaining anatomical landscape are reduced or avoided. However, the flaring arrangement ensures that a fluid flow from the proximal end to the distal end is not significantly impaired. Furthermore, as indicated in FIG. 9C, the proximal end 16 and the distal end 17 are inverted. Such flaring of the second closed loops facilitates the insertion of e.g. a valve assembly into the body 12 by forming a chamfering surface.

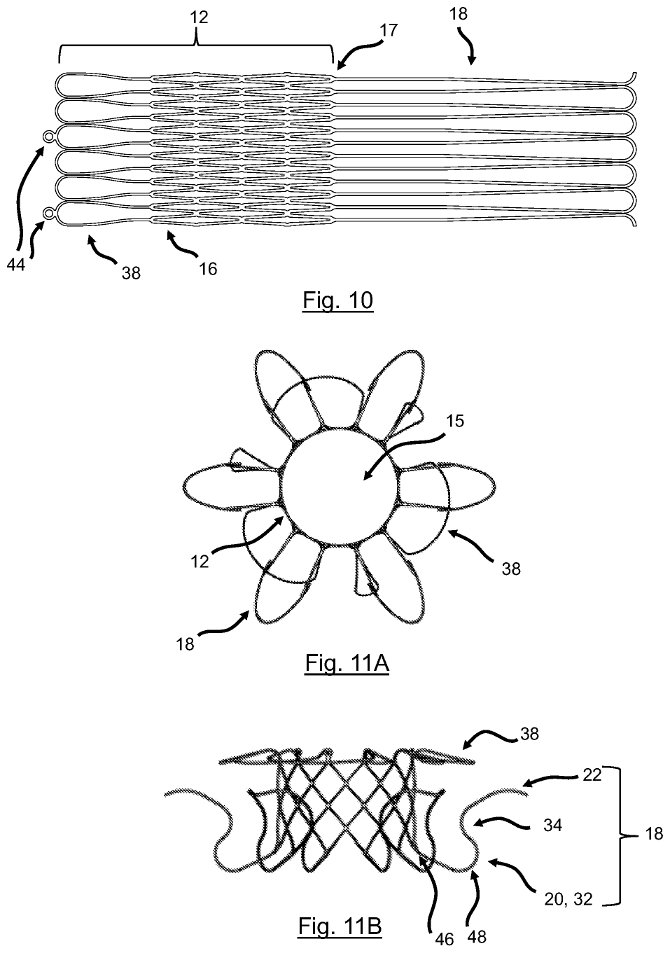

In FIG. 10 a schematic representation of a stent device 10 according to the invention after laser cutting and prior to heat forming is shown as an alternative to the embodiment depicted in FIG. 3B. In this embodiment, eyelets 44 are provided on a proximal end 16 or proximal tip end of the second loops 38 of the stent body 12, which enable that the stent device 10 may be secured to a delivery system for insertion into the anatomic region of interest. Accordingly, said eyelets 44 may also be used after insertion for release and final deployment of the stent device 10. In this embodiment a total of two eyelets 44 is provided, which are arranged on the closed loops 38 so as to be at opposing sides of the stent device 10 in a radial direction in the assembled state. However, the arrangement and number of eyelets 44 may be chosen as desired. Furthermore, the eyelets 44 have a circular or rounded shape so as to facilitate fixation to and release from a delivery system. Alternatively, the eyelets may also comprise other shapes, preferably a symmetrical design, such as a polygonal or ellipsoid shape.

In FIGS. 11A and 11B a schematic top view and a perspective side view of a stent device 10 having an alternative support arm configuration is respectively shown.

In this configuration, a flexible region “in between” having a tapered section is not provided, as best shown by a direct comparison with the embodiment according to FIG. 9A, wherein constrictions are schematically indicated by sharp v-shaped notches. Instead, according to the embodiment of FIG. 11A, each support arm 18 comprises a tapered section, which gradually extends essentially from the concave portion 34 towards the second support region 22, i.e. towards the proximal tip end of the support arm 18. The tapering is provided as an essentially triangular or conical or ellipsoid shape and comprises a rounded portion at the proximal tip end, e.g. an end portion or loop portion, of the second support region 22, so as to reduce the risk of tissue damage.

At the same time, a spring function is maintained between the second support region 22 and the first support region 20, which is improved by a convex portion 32, which defines a shoulder or curvature of the first support region 20 with an adjacent concave portion 34 and which is linked to the distal end of the stent body 12, as shown in the perspective side view of FIG. 11B. The shoulder has a radius 46 at the distal end of the shoulder that is larger than a radius 48 at the proximal end of the shoulder. Accordingly, a wider angle is provided at the distal end of the stent body 12, which together with the radial extension of the convex portion 32 ensures that the likelihood of fractures occurring between the support arms and the stent body 12 is reduced and that forces acting upon the stent device 10 and the support arms 18 may be better distributed.

The smaller radius 48 at the proximal end of the shoulder furthermore ensures, together with the adjacent concave portion 34, that an improved adaptation to the annulus region and ventricular anatomy is provided. Furthermore, the radial extension of a radially outermost point of the convex portion 32—in radial direction—lies between a radially innermost point of the concave portion 34 and a proximal tip of the second support region 22, such that the proximal tip end of the second support region 22 has the largest radial extension of all sections of each support arm 18. Thereby, while providing an improved fitting or adaptation to the anatomical annulus region, a contacting surface is increased at the atrial portion and a wider support is provided without detrimentally affecting the remaining native structure of the annulus region.

In other words, the wider extension of the supporting regions of each support arm provide a larger engaging or interacting surface, while the particular configuration of the convex portion 32 and concave portion 34 as well as the radial extension of the second support region 22 ensures that an improved spring function is established with an improved absorption and distribution of forces acting upon the stent device.

Also shown in this embodiment is the mesh-shape of the body, which is here formed by a lattice of a plurality of diamond-shaped cells that are directly connected to each other and are essentially equal in size and/or shape. As outlined in the above, the diamond shape has the advantage of providing a substantially equal stress and strain resistance in the axial and circumferential direction and that the amount of deformation and strain applied during manufacturing may be reduced so as to increase the stability of the body. Furthermore, a required pliability may be retained, e.g. by varying thicknesses towards the proximal and/or distal ends.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 Stent device

### 12 Body

### 13 Strut

### 14 Cell

15 Inner channel

### 16 Proximal end

### 17 Distal end

### 18 Support arm

20 First support region
22 Second support region
24 Flexible region

### 26 Annulus

27 Native cusp or leaflet
28 Ventricular portion
30 Atrial portion
32 Convex portion
34 Concave portion

### 36 Linking arm

38 Proximal end portion or second closed loops
40 Fixation means or window
42 Delivery system

### 44 Eyelet

46 Distal radius
48 Proximal radius

1. Stent device (10) for a prosthetic heart valve, comprising:
a mesh-shaped body (12) extending in an axial direction, said body (12) being configured to fit an orifice and defining an inner channel (15) for providing a passageway from a proximal end (16) to a distal end (17) of the body (12), and
at least three outer support arms (18) extending from the body (12) from the distal end (17) of the body (12) towards the proximal end (16), each support arm (18) comprising a first support region (20) at the distal end (1 7), and a second support region (22) at the proximal end (16), wherein the second support region (22) extends radially outwards in the deployed state,
wherein each support arm (18) comprises a flexible region (24) between the first support region (20) and the second support region (22), which is formed as a tapered section of the support arm (18) in an axial direction, and/or
wherein each support arm (18) is tapered towards the proximal end (16).

2. Stent device (10) according to claim 1, wherein the body (12) is configured to fit an annulus (26) of the heart valve, wherein the flexible region (24) is adapted to conform to the annulus (26), the first support region (20) is adapted to conform to the ventricular portion (28) of the annulus (26), and/or the second support region (22) is adapted to conform to the atrial portion (30) of the annulus (26).

3. Stent device (10) according to any of the preceding claims, wherein the body (12) comprises an essentially tubular or cylindrical shape.

4. Stent device (10) according to any of the preceding claims, wherein each support arm (18) is formed as a closed loop.

5. Stent device (10) according to claim 4, wherein the closed loop extends beyond the proximal end (16) of the body (12) and/or comprises a rounded and/or tapered proximal end.

6. Stent device (10) according to claim 4 or 5, wherein the closed loop defines a profile having a convex portion (32) and a concave portion (34) in a longitudinal section of the support arm (18) and wherein the convex portion (32) defines the first support region (20), wherein the concave portion (34) is preferably adjacent to the convex portion (32).

7. Stent device (10) according to claim 6, wherein each support arm (18) comprises the flexible region (24) between the first support region (20) and the second support region (22) and wherein the concave portion (34) defines the flexible region (34).

8. Stent device (10) according to claim 6 or 7, wherein a radial extension of a radially outermost point of the convex portion (32) is larger than a radially innermost point of the concave portion (34) and/or wherein a radially outermost point of the convex portion (32) lies between a radially innermost point of the concave portion (34) and a proximal tip of the second support region (22).

9. Stent device (10) according to any of claims 6 to 8, wherein the profile is formed as an inverted S-shape, sine wave shape, N-shape, or M-shape in an axial direction and/or in a radial direction.

10. Stent device (10) according to any of the preceding claims, wherein each support arm (18) is linked to the body (12) via at least one linking arm (36) formed by a curvature of the first support region (20).

11. Stent device (10) according to claim 10, wherein each support arm (18) is linked to the body (10) via two linking arms (36).

12. Stent device (10) according to claim 10 or 11, wherein the curvature comprises an angle of more than 90° and/or defines a rounded shoulder, said shoulder preferably having a distal radius (46) and a proximal radius, wherein the distal radius (46) is larger than the proximal radius (48).

13. Stent device (10) according to any of the preceding claims, comprising an uneven number of support arms (18), preferably 5, 7, or 9 support arms (18), or a multitude/multiple of two and/or three support arms (18), said support arms (18) being adapted to a tricuspid valve or mitral valve.

14. Stent device (10) according to claim 13, comprising six support arms (18) and being configured for a tricuspid valve.

15. Stent device (10) according to any of the preceding claims, wherein the circumferential spacing between the support arms (18) is asymmetric or symmetric and/or is adapted to a tricuspid valve or mitral valve.

16. Stent device (10) according to any of the preceding claims, wherein the mesh shape of the body (12) comprises a droplet shape, a diamond shape, or an essentially oval shape.

17. Stent device (10) according to any of the preceding claims, wherein the mesh-shape of the body (12) is formed by a lattice of a plurality of diamond-shaped cells (14) that are directly connected to each other or are connected via struts (13), said cells (14) preferably being essentially equal in size and/or shape.

18. Stent device (10) according to any of the preceding claims, wherein a portion (38) of the proximal end (16) of the body (12) extends radially outwards.

19. Stent device (10) according to claim 18, wherein the portion (38) of the proximal end (16) of the body (12) extends between 70° and 110° with regard to the axial direction of the body (12).

20. Stent device (10) according to claim 18 or 19, wherein the portion (38) is defined by a plurality of second closed loops, which are preferably arranged in a circumferentially staggered formation with regard to the support arms (18) arranged at the distal end (17).

21. Stent device (10) according to claim 20, wherein the portion (38) comprises at least one eyelet (44) for securing the stent device (10) to a delivery system, preferably at least two eyelets (44), each of the at least one eyelet(s) (44) being arranged at a respective second closed loop, preferably at every second or third second closed loop and/or at the proximal end (16) or radially outermost end of the second closed loop.

22. Stent device (10) according to any of the preceding claims, wherein the body (12) and the plurality of support arms (18) are formed as a single piece and/or as a wire frame.

23. Stent device (10) according to any of the preceding claims, wherein the body (12) or the passageway defined by the body (12) comprises an inner diameter between 29 mm and 36 mm, preferably about 30 mm or about 35 mm.

24. Stent device (10) according to any of the preceding claims, wherein at least the second support region (22) of the supporting arms (18) and/or the proximal end (16) of the outer body (12) are covered with a foil of a liquid impermeable or semi-impermeable material so as to form a cuff between the support arms (18) and the body (12) and/or between the support arms (18).

25. Stent device (10) according to any of the preceding claims, wherein the body (12) comprises at least two or at least three fixation means or windows (40) for receiving a valve assembly or wherein the cells of the mesh-shaped body (12) are configured for receiving and fixation of a valve assembly.

26. Prosthetic heart valve, comprising a stent device (10) according to any of the preceding claims and a valve assembly arranged within the inner channel (15) and/or at a proximal (16) or distal end (17) of the body (12) and being secured to the body (12) by means of fixation means or windows (40) or direct fixation to one or more cells of the mesh-shaped body (12).

27. Prosthetic heart valve according to claim 26 configured for replacing a tricuspid valve or a mitral valve.

28. Delivery system, comprising the stent device according to any of the preceding claims in a collapsed state.

29. Method for replacing a tricuspid valve or mitral valve, comprising the steps of:
providing a stent device according to any of the claims 1-25 in a collapsed state in a delivery system,
percutaneously introducing the stent device into a tricuspid valve or mitral valve region of a patient via said delivery system, such that the distal end of the body is at a ventricular portion and the proximal end of the body is at an atrial portion and the body and support arms straddle the annulus, and
deploying the stent device by expanding the stent device, such that the flexible region conforms to the annulus and the second support region of the outer support arms conform to the atrial side.

30. Method of producing a stent device according to any of the claims 1-25, comprising the steps of:
laser cutting the body and support arms from a metallic memory material;
heat forming the body and support arms, so as to provide a predefined shape of the body and support arms; and
collapsing the body and support arms.

31. Method according to claim 30, wherein the stent device is made from a single piece.
